(19) United States
(12) Patent Application Publication
(10) Pub. No.: US 2026/0227377 A1
(43) Pub. Date: Aug. 6, 2026

(54) SYSTEMS, METHODS, AND COMPUTER-READABLE MEDIA FOR DETECTION AND MONITORING OF SOIL CARBON SEQUESTRATION

(71) Applicant: THE REGENTS OF THE UNIVERSITY OF COLORADO, A BODY CORPORATE, Denver, CO (US)

(72) Inventors: Jason C. NEFF, Boulder, CO (US); Gregory L. WHITING, Boulder, CO (US); Evan THOMAS, Boulder, CO (US); Taylor SHARPE, Boulder, CO (US)

(21) Appl. No.: 19/151,239

(22) PCT Filed: Jan. 26, 2024
(86) PCT No.: PCT/US2024/013187

§ 371 Date: Jul. 25, 2025

Related U.S. Application Data
(60) Provisional application No. 63/441,707, filed on Jan. 27, 2023.

(51) Int. Cl. G01N 33/24

(52) U.S. Cl. CPC G01N 33/24

(57) ABSTRACT

A system for detection and monitoring of soil health comprises a plurality of carbon sensor devices, each being configured to be planted in soil at a plurality of sites, a network interface configured to aggregate outputs generated by the plurality of carbon sensor devices, one or more processors, and one or more computer-readable media. Executable instructions are saved on the one or more media and, when executed by the one or more processors, configure the system to perform preprocessing the aggregated outputs, analyzing, by a machine learning algorithm, the preprocessed outputs with environmental parameters, estimating a soil parameter of the plurality of sites, and estimating a local soil parameter for at least a subset of the plurality of sensor sites based on the estimated soil parameter.

### CROSS-REFERENCE TO RELATED APPLICATIONS

This application claims the benefit of and priority to U.S. Provisional Patent Application Ser. No. 63/441,707 filed on Jan. 27, 2023, and entitled “INTEGRATED SYSTEM FOR THE DETECTION AND MONITORING OF SOIL CARBON SEQUESTRATION,” which is incorporated herein by reference in its entirety.

### GOVERNMENT RIGHTS

This invention was made with government support under grant number 2019-05291 awarded by the U.S. Department of Agriculture. The government has certain rights in the invention.

### FIELD

The present disclosure relates generally to systems, methods, and computer-readable media for monitoring soil carbon sequestration, and particularly for automatically detecting and monitoring carbon in-situ in the soil based on a plurality of carbon-related sensors.

### BACKGROUND

The United States agricultural system was responsible for 646 million metric tons of CO₂ equivalent production in 2019. Soil carbon sequestration may be capable of removing up to 250 million metric tonnes of carbon per year. With a potential market price between 15 and 30 dollars per tonne and emerging systems for the exchange of carbon sequestration credits, the soil sequestration market in the US alone can be worth up to $3.75-7 billion per year. If monitoring and validation approaches are valued at 5-15% of the total market (consistent with current pricing), the potential market for soil sequestration monitoring is in the range of $175-300 million per year in the United States.

Though the soil carbon sequestration market is large, current methods for monitoring carbon stabilization have multiple limitations, with growing number of critics of the current validation approaches. Soil organic matter is highly variable across fields and soil depth. Changes in soil carbon stabilization are small relative to the size of the overall stock of below-ground carbon, making small-scale changes in carbon during sequestration notoriously hard to measure. Field scale soil organic carbon (SOC) measurements are made using destructive sampling of soil taken from multiple depths and locations on a farmer's field. These samples are processed, combusted and the CO₂ evolved from the soil is converted back into an estimate of SOC concentrations. The process is labor intensive, requires advanced analytical facilities and typically generates large field-scale uncertainty terms. The uncertainty associated with these field-based methods is high. Changes in soil carbon storage over time during sequestration are often close to the analytical (measurement) and ecological (natural variability) errors associated with field scale carbon stock estimates making validation of sequestration problematic. A wide range of remote sensing approaches using imaging spectroscopy have been proposed but these are prone to low accuracy and high uncertainty since most carbon is stabilized in subsurface layers invisible to remote sensing approaches without destructive sampling of the soil surface requiring that remote sensing approaches be coupled to simulation models in order to estimate soil carbon changes over time.

Given that current methods for field monitoring of soil carbon sequestration are problematic for use at scale, validating carbon markets uses soil sequestration simulation models built from semi-empirical relationships between management, soil properties, physical variables, and SOC content or management based ‘look-up tables’ that assume a degree of carbon stabilization associated with changing management approaches. While simulation models are important tools, they are prone to high degrees of uncertainty and for the reasons described above are difficult to validate. These results in a rapidly developing market focused on the management of potentially millions to billions of dollars of stabilized soil carbon are based on a striking degree of uncertainty. Moreover, stabilized SOC has a finite residence time ranging from months to decades depending on the nature and location of the stabilization. Carbon sequestered for months in soil has no value to climate mitigation, whereas carbon stabilized for decades or more could be a viable tool in mitigation strategies.

Virtually all the existing monitoring and validation approaches for soil carbon sequestration have significant issues that threaten to undercut the market or can result in lower-than-expected carbon pricing (due to the uncertainty in measurement and/or permanence). Given these issues, there is a pressing need for new monitoring approaches to below-ground carbon stock changes over time. Moreover, such technologies can also aid in the monitoring of a broader range of soil heath and nutrient management issues.

The subject matter claimed herein is not limited to aspects that solve any disadvantages or that operate only in environments such as those described above. Rather, this background is only provided to illustrate one exemplary technology area where some aspects described herein may be practiced.

### BRIEF SUMMARY

Disclosed aspects include a system for detection and monitoring of soil carbon sequestration comprising a plurality of sensors configured to be disposed in soil at a plurality of sites. The system further comprises one or more processors and one or more computer-readable media having stored thereon executable instructions that when executed by the one or more processors configure the computer system to identify a predicted soil parameter for at least a subset of the plurality of sensor sites.

According to various aspects of the present disclosure, provided is a carbon sensor device for sensing changes in carbon sequestration in soil. The carbon sensor device includes an elongated body configured to be planted into soil along a longitudinal direction thereof and a plurality of arrays of sensors fixedly attached to the elongated body. An array of sensors includes a carbon dioxide sensor, a moisture sensor, and a temperature sensor. The plurality of arrays of sensors are attached to different positions along the longitudinal direction.

According to various aspects of the present disclosure, provided is a system for detection and monitoring of soil health. The system includes a plurality of carbon sensor devices, of which each is described above and is configured to be planted in soil at a plurality of sites, a network interface configured to aggregate outputs generated by the plurality of carbon sensor devices, one or more processors, and one or more computer-readable media. Executable instructions are saved on the one or more media and, when executed by the one or more processors, configure the system to perform preprocessing the aggregated outputs, analyzing, by a machine learning algorithm, the preprocessed outputs with environmental parameters, estimating a soil parameter of the plurality of sites, and estimating a local soil parameter for at least a subset of the plurality of sensor sites based on the estimated soil parameter.

According to various aspects of the present disclosure, provided is a computer-implemented method for estimating soil health. The method includes aggregating outputs generated by a plurality of carbon sensor devices, of which each is described above and is configured to be planted in soil at a respective one of a plurality of sites, preprocessing the aggregated outputs, analyzing, by a machine learning algorithm, the preprocessed outputs with environmental parameters, estimating a soil parameter of the plurality of sites, and estimating a local soil parameter for at least a subset of the plurality of sensor sites based on the estimated soil parameter.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific aspects which are illustrated in the appended drawings. Understanding that these drawings depict only typical aspects and are not therefore to be considered to be limiting in scope, aspects will be described and explained with additional specificity and detail through the use of the accompanying drawings.

FIG. 1 shows a graphical illustration showing carbon flow through plant/soil system according to various aspects of present disclosure.

FIG. 2 shows a schematic diagram for a carbon sensor device according to various aspects of present disclosure.

FIG. 3 shows a schematic diagram for a system with carbon sensor devices according to various aspects of the present disclosure.

FIG. 4 shows a flowchart of a method for detecting and monitoring soil health according to various aspects of the present disclosure.

### DETAILED DESCRIPTION

The following discussion now refers to a number of devices, systems, methods, and method acts that may be performed. Although the method acts may be discussed in a certain order or illustrated in a flow chart as occurring in a particular order, no particular ordering is required unless specifically stated, or required because an act is dependent on another act being completed prior to the act being performed.

In various disclosed aspects, an array of sensors utilizes low-cost sensors in complex and variable soil settings, an advanced data assimilation and analytics algorithms, and incorporation of sensors that have been designed with cutting edge material science and printed electronics technology. In various aspects, a reliable approach may be generated to address two high priority national and global needs: 1) soil carbon monitoring of soil health that leads to commercial applications, and 2) improvement in the monitoring of soil carbon sequestration which currently largely relies on techniques that are prone to large uncertainties and/or high costs. Based on the present disclosure, current soil carbon sequestration systems and methods may make this type of land modification a trusted and central tool not only for mitigating the rise of greenhouse gases but ultimately benefiting farmers, food producers, and society as a whole.

The conventional measurement of CO₂ flux from soils to the atmosphere is carried out using a sealed chamber in which the rate of CO₂ accumulation is a measure for the net flux of CO₂ from soils. Soil CO₂ flux can also be modeled using diffusion equations driven by observations of the vertical profile of CO₂ through the soil and about the soil surface; however, this approach is limited by the cost of each sensor (typically greater than $1,000 per sensor), data collection system (often greater than $2,000) and the ancillary data required for accurate flux calculations. Decomposition is a microbially mediated process and is strongly and predictably sensitive to changes in temperature and moisture and our proposed design includes a combination of CO₂, temperature and moisture sensors to drive the analytical platform described below.

The decomposition and physical stabilization of soil organic carbon (SOC) operate simultaneously in soils. SOC is a chemically complex array of materials ranging from simple sugars to long-chain aliphatic or aromatic carbon compounds. These different compound classes are attacked by different enzyme produced by a range of different organisms and are decomposed to CO₂ at different rates (that range from hours to years). Stabilization processes are equally complex and are the result of the inherent chemical resistance of some plant materials, the chemical association of SOC with mineral surfaces, and the physical protection of SOC in macro structures such as aggregates.

The concept of carbon sequestration is poorly defined in part because of the varied time horizons for carbon stabilization or release. Some carbon (such as wood) is stabilized for timescales of months to a year (or years) because of the inherent resistance to degradation. Chemically stabilized carbon on mineral surfaces can remain in place for decades to a century or more and physically protected carbon in structures such as aggregates can store carbon for years to decades. Carbon sequestration is therefore contingent on the time frame of contractual or policy related requirements for the duration of stabilization. In principle these should require decadal or multi-decadal storage which then necessitates some type of physical or chemical/mineral stabilization process.

Soil CO₂ fluxes measure the combined effect of both the decomposition and stabilization processes with plant root CO₂ production. Over multiple years & with excellent separation of the root contribution, and accurate estimation of carbon inputs from plants, the net change in CO₂ flux would represent the net sequestration rate for a field. In practice, this measurement is near impossible to obtain. Disclosed aspects use proxy measures of key decomposition process rates and stabilization pathways to build a multi-modal approach to the monitoring of carbon sequestration in a field. The stabilization and decomposition sensors described below provide two crucial data inputs that may be used in combination with CO₂ fluxes and analytical modeling to create a comprehensive picture of the state of carbon stability in a field setting.

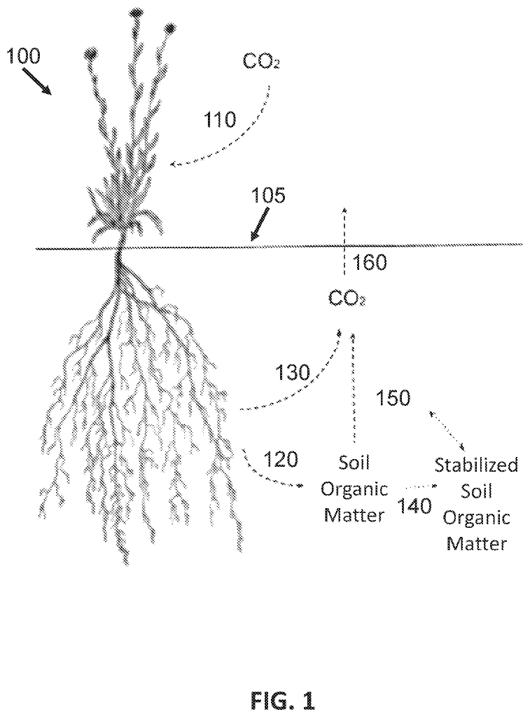

FIG. 1 shows a graphical illustration showing a carbon flow through a plant/soil model according to aspects of present disclosure. As illustrated, a plant 100 may be planted into soil: the top portion of the plant 100 is positioned over the top surface 105 of the soil, and the bottom portion of the plant 100, or the roots are positioned below the top surface 105 of the soil. Even though the top portion of the plant 100 exhales CO₂ at night, it generally inhales CO₂ at step 110 from the air to synthesize CO₂ and water into glucose and O₂ at chloroplasts. This process is called photosynthesis. The fixation of CO₂ by the top portion of the plant 100 from the air may be measured at the harvest period by the biomass of the plant 100. Thus, the aboveground carbon cycling processes, which happen seasonally in agricultural fields (plant growth), may be monitored directly through harvests or approximated with relatively good accuracy from remote sensing platforms.

On the other hand, under the top surface 105 of the soil, when carbon enters the soil, CO₂ may be produced from the decomposition of soil organic matter (heterotrophic respiration) at step 150 and the production of CO₂ in the roots (autotrophic respiration) of the plant 100 at step 130. As such, production of CO₂ within the soil may be from heterotrophic respiration by the roots and autotrophic respiration by the soil organic matters and the stabilized soil organic matters.

Further, the carbon may be decomposed and released with CO₂ and this process is strongly dependent on chemical compositions of the incoming materials and activities of soil microorganisms which in turn is strongly regulated by temperature and moisture conditions.

The soil organic matters may utilize CO₂ to increase its biomass at step 120. Any carbon that is not decomposed can be stabilized into soil physical structures such as aggregates or onto the surfaces of minerals at step 140. It is the physical processes that are responsible for longer-term (decades or more) carbon sequestration, but these stabilization pathways may be nested within the larger context of carbon flow and loss from plant to soil systems. In the stabilized soil organic matter, on the other hand, may be protected from decomposition and may contain CO₂ for a comparatively longer period than the general soil organic matters. In other words, decomposition of the stabilized soil organic matter may be performed at step 150 in a slow pace, while decomposition of the general soil organic matter may be made at step 150 in a relatively faster pace.

The small net difference between soil CO₂ fluxes and the net primary production (total carbon produced in biomass) of the plant 100 determines whether an agricultural system is accumulating or losing carbon. Carbon sequestration is challenging to monitor because of the size of the gross fluxes of carbon into and out of soils, the large background pool of stabilized carbon in the soils and the heterogeneity of processes at field scales. Despite these issues, portions of the agricultural carbon system are easier to characterize than others.

In most field systems, it is possible to develop a reasonably accurate estimate of carbon inputs to soils from existing technology and knowledge of above and below-ground carbon inputs from plants. Once plant carbon enters the soil, there are multiple possible fates.

A central concern in current carbon sequestration monitoring approaches is the lack of field based physical assessments that can be used to build confidence (or simply to test) model outputs. The primary advances in carbon monitoring in recent decades has been in CO₂ flux measurements using soil surface chambers, soil CO₂ concentration monitoring and/or eddy correlation techniques that monitor concentration changes in upward and downward flows of CO₂ during turbulent exchange in the boundary layer above the field. None of these methods individually are capable of detecting the small amounts of carbon that are stabilized during sequestration. Soil CO₂ flux measurements, however, can be an important constraint on below ground processes but need to be coupled to models to estimate the contributions of CO₂ from roots or soil organic matter decomposition. These measurements are also difficult and expensive to make and require a coupling with biophysical models to estimate the production pathways of CO₂. Moreover, without additional information on the rates and dynamics of decomposition and stabilization, the model output will be relatively unconstrained and sequestration estimates will remain elusive.

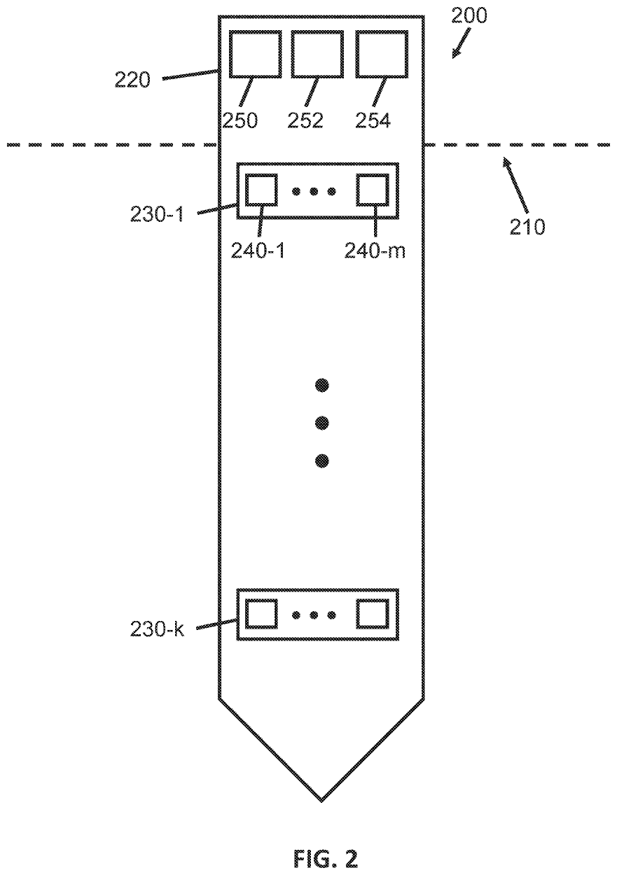

Disclosed aspects address this issue with an integrated monitoring device or a carbon sensor device 200, as illustrated in FIG. 2. The carbon sensor device 200 may be placed at a position in the region of interest so that it provides sensing outputs and the carbon sequestration may be measured and estimated based on the in-situ sensing. The carbon sensor device 200 may have an elongated main body 220, which provides structural support for the carbon sensor device 200. The elongated main body 220 may have sufficient sturdiness so that, when the carbon sensor device 200 is inserted into the soil, the carbon sensor device 200 is able to maintain its structure within the soil. Further, since the carbon sensor device 200 may stay in the soil for months or years, the elongated main body 220 may be chemically stable so that no substantial decomposition or decay is occurred within the soil for the lifetime of the carbon sensor device 200.

The length of the carbon sensor device 200 or the elongated main body 220 may be less than or equal to 1 meter, 80 centimeters (cm), 60 cm, 50 cm, or 30 cm. This list of lengths is provided as examples but is not limited thereto. The length of the carbon sensor device 200 may depend upon requirements. For example, in a case where the region of interest for carbon sequestration is within 30 cm from the top surface 210 of the soil, the part of the carbon sensor device 200, which is inserted into the soil may be greater than the region of interest. That means, the length of the part may be greater than or equal to 30 cm, such as 35 cm or 40 cm, and the portion of the elongated main body 220, which is above the top surface 210 of the soil, may be greater than or equal to 10 cm, 20 cm, 30 cm, 40 cm, or 50 cm. The top portion of the carbon sensor device 200 may be prespecified based on data collection requirements. For example, to be seen with ease, the top surface of the top portion of the carbon sensor device 200 may be above the plants nearby. Thus, the total length of the carbon sensor device 200 may be a sum of the bottom portion and the top portion.

The elongated main body 220 may have a circular, rectangular, triangular, or any other shape when cut perpendicular to the longitudinal direction. Further, the bottom portion of the elongated main body 220 may have a sharp or pointy shape so that, when the carbon sensor device 200 is planted into the soil, the elongated main body 220 may be easily inserted into the soil.

The carbon sensor device 200 may further include a plurality of arrays of sensors 230-1-230-k, where k may be two or more. The plurality of arrays of sensors 230-1-230-k may be affixed to the elongated main body 220 along the longitudinal direction of the elongated main body 220. In other words, the plurality of arrays of sensors 230-1-230-k may be arranged vertically. The bottom sensor 230-k may be positioned at or above the depth (e.g., 50 cm, 30 cm, or 1 m) of the region of interest, and the top array of sensors 230a may be positioned below the top surface 210 of the soil. This vertical configuration of the plurality of arrays of sensors 230-1-230-k may yield vertical concentration gradients across the longitudinal direction to estimate CO₂ fluxes from the soil to the air.

Each array of sensors 230-1-230-k may include carbon-related sensors 240-1-20-m, where m may be greater than two. Each array may be a mere combination of carbon-related sensors 240-1-20-m or an integrated circuit, on which each carbon-related sensors 240-1-240-m are electrically coupled. In an aspect, carbon-related sensors 240-1-240-m may be three-dimensionally printed on a flexible substrate, which can be easily attached to the elongated main body 220.

Each array of sensors 230-1-230-k may include a low-cost CO₂ sensor coupled to low-cost microcontrollers (not shown), enabling data collection in the carbon sensor device 200. In an aspect, simple modifications to ruggedize NDIR CO₂ sensors may be made for use in soil and aquatic environments. The initial low cost of these sensors reflects intended use as indoor sensors, but they have been shown to be reliable and to perform well with respect to industrial gas sensors.

In an aspect, the carbon-related sensors 240-1-20-m may include a temperature sensor and a moisture sensor because prior work estimating subsurface CO₂ fluxes using similar strategies found varying degrees of temperature, humidity, and soil water content. One difficulty in applying the gradient method to estimate gas flux may be that the diffusivity of soil can change as a function of soil moisture content. In some aspects, it is therefore necessary to measure the soil moisture at each depth.

Low-cost capacitive soil moisture sensors have been the focus of the conventional art, where the designers have generally used a timer IC to convert a capacitive reading to an analog value or have taken advantage of newly-ubiquitous touch sensor interfaces. Both of these approaches have significant advantages for large-scale deployments, including improved corrosion resistance, sensing surface geometric variability, and very low component costs. Various configurations of these low-cost moisture sensing surfaces may be integrated into the carbon sending device 200 and be deployed across a wide range of soils by evaluating the need for soil-specific calibrations. The moisture sensor may be suitable for long-term, autonomous measurements of soil carbon flux, supporting advanced modeling and data assimilation techniques. The low cost of each sensor may aid adoption of the carbon sensor device 200 in soil carbon flux monitoring, as improved spatial and temporal resolution may provide modelers with the tools needed to accurately measure carbon fixation processes.

In another aspect, the carbon-related sensors 240-1-240-m may include a decomposition sensor and a carbon accumulation sensor. The decomposition sensor may use a combination of a material that degrades over time based on type and intensity of microbial activity, eventually exposing a water soluble conductor which rapidly degrades causing an open circuit. The status of this fuse-like sensor (open or closed circuit) may be evaluated and reported by simple electronic circuits. In various aspects, the decomposition sensor may be built based on 1) parallel groupings of fuses to provide a multi-level response; 2) capacitive or resistive approaches based on the degradation of conductive substrate causing either a drop in resistance, or decrease in capacitance due to reduction of electrode area, which in both cases may provide a continuous rather than discrete signal; 3) organic electrochemical transistors with degradable channels that may give a continuous signal proportional to the rate of decomposition of a particular material of interest. For these three sensor types, materials that are representative of the types of chemical compounds present in plant and soil organic matter (e.g., polysaccharides, lignins, waxes, and carbohydrates), may be chosen as the encapsulant, substrate, and binder.

The accumulation sensor may be used for soil carbon accumulation. In the accumulation sensor, surfaces may be created to mimic the soil surfaces where long-term carbon accumulation occurs. These surfaces may be metal or silicon oxides or other organic materials. Soluble and solid organic compounds may accumulate on the accumulation sensor over time, triggering a signal that is proportional to the rate of carbon accumulation on surfaces of the accumulation sensor. In various aspects, the accumulation sensor may have capacitive structures, with carbon accumulation causing a change in dielectric constant of the sensor surfaces, leading to a capacitive signal proportional to the amount of accumulated carbon-based material. The accumulation sensor may then serve as a direct proxy for carbon accumulation/sequestration in the soil.

Now referring back to FIG. 2, the carbon sensor device 200 may include a memory 250, which is to store sensor outputs from the plurality of arrays of sensors 230-1-230-k. The sensor outputs are generally analog and digitized to generate digital sensor outputs. The memory 250 may store the digital sensor outputs (hereinafter “sensor outputs” or just “outputs”). The capacity of the memory 250 may be sufficient to store the outputs for at least two collection periods or more. The outputs may be collected or aggregated daily, weekly, biweekly, monthly, bi-monthly, quarterly, semi-annually, or annually. Thus, when the outputs are collected annually, the memory 250 may have a capacity to store at least two years of the outputs. In an aspect, each sensor may have a collecting period different from each other.

Further, the memory 250 may store the outputs from each carbon-related sensors with their identifications. For example, the memory 250 stores the identification of each array and the identification of each sensor so that, when the outputs are collected, the location within the elongated main body 220 may be identified.

In an aspect, the memory 250 may be positioned in each array with the carbon-related sensors 240-1-240-m. In this instance, the memory 250 of each array may store outputs from the carbon-related sensors 240-1-240-m in the array. Further, the memory 250 of each array may also include an identification of each array so that outputs saved in memories 250 of other arrays may be distinguished from each other.

The carbon sensor device 200 may further include a network interface 252, which may communicate with a collecting device by following wireless communication protocols, which may be near field communication (NFC), Bluetooth, Wi-Fi, and the likes. The network interface 252 may be used to transfer the outputs saved in the memory 250. This wireless communication may be one factor to lower the costs than existing manual-intensive collection approaches.

Furthermore, the carbon sensor device 200 may include a power source 254 (e.g., a battery), which supplies power to the plurality of arrays of sensors 230-1-230-k. Since each sensor needs nether much power nor constant sensing, the power required for the carbon sensor device 200 may be minimal and does not have to be continuously provided. In an aspect, the power source 254 may be wired to a power outlet, which continuously provides power. In another aspect, the power source 254 may be a power generator based on the sun, wind, or any renewable energy source. In still another aspect, the power source 254 may be integrated into the network interface 252. In this case, radiofrequency signals may activate the network interface 250 and the network interface 250 may convert the radiofrequency signals to energy to supply power to the to the plurality of arrays of sensors 230-1-230-k.

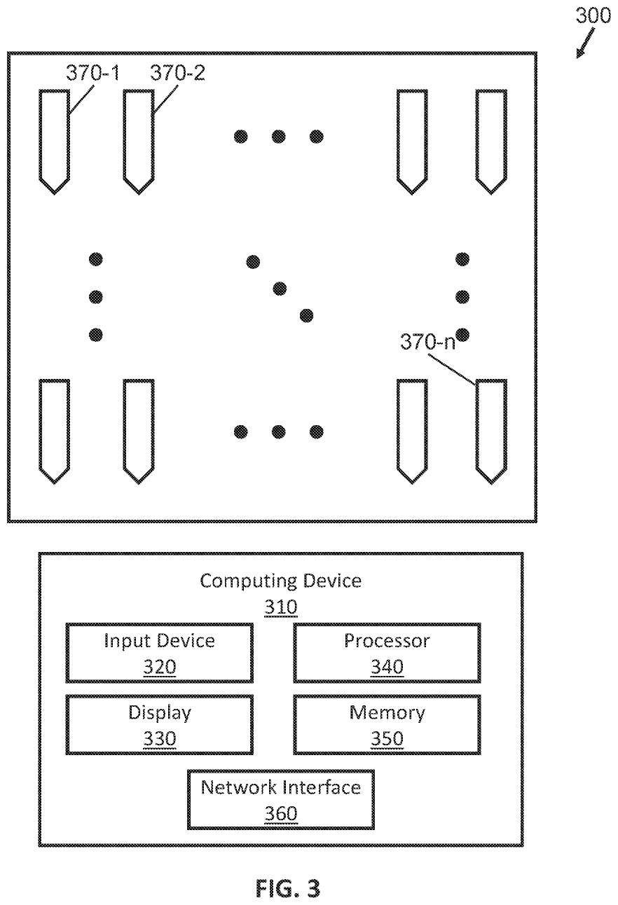

Now turning to FIG. 3, illustrated is a plurality of carbon sensor devices 370-1-370-n (e.g., the carbon sensor device 200 of FIG. 2) may be planted into the soil in the region of interest of a plot of land. The respective places of the plurality of carbon sensor devices 370-1-370-n may be places, which may represent the nearby area. As such, illustrated by FIG. 3 is a system 300 which combines remote sensing with in-situ monitoring of soil CO₂ coupled using data assimilation algorithms to a mechanistic model of soil carbon decomposition and stabilization.

Prior to installation of the carbon sensor devices 370-1-370-n in a field, a sensor placement design algorithm may be developed in a field that will minimize costs (e.g., minimum number of the carbon sensor devices 370-1-370-n) and optimize field level carbon measurements. This placement algorithm may be generated through the use of remote sensing and geospatial data and field level assessment when needed. Individual station data flows may be composited and extrapolated to field level carbon sequestration estimates using similar approaches.

A common challenge with any in-situ sensors is that they are not designed for long-term use. This is primarily because of fouling and signal drift that attenuate and ambiguate the signal as well as natural variability within and between sites. This limitation may be addressed through long-term characterization of in-situ parameters, transmission of the site-level sensor data to an online data assimilation platform, combination of the data from a network of sensors with additional data sets including remote sensing sources, and analysis of parameters and change detection with mechanistic and/or machine learning models.

Through this method, the site-level parameter estimates are improved by incorporating data from a network of sensors and data sources beyond the performance attainable when examining each sensor individually.

Disclosed aspects leverage a sensor output fusion approach to learn from varying types of sensors and sites to improve the sensitivity and specificity of parameter estimates. This approach of unified hardware and complex statistical learning tools allows a rapid deployment and adoption of Internet of Things (IOT) solutions. Disclosed aspects include the capability to aggregate in-situ sensor data, transmit it, combine it with other environmental data sources (like remote sensing, weather, altitude, etc.) and a machine learning model to then generate site-level predictions and parameter estimates relating to soil health in a way that is more accurate than the sensor data just by itself.

For example, the system 300 may utilize a change point detection machine learning algorithm. As non-limiting examples, one common change point detection algorithm for sensor data is the cumulative sum (CUSUM) algorithm. The CUSUM algorithm compares the current sensor reading to a running average or expected value, and if the difference exceeds a certain threshold, a change point is detected. The threshold can be set based on the desired sensitivity of the detection and the expected noise level in the sensor data. The CUSUM algorithm can also be combined with other techniques such as the likelihood ratio test to increase its robustness. Another algorithm that can be used may be the Bayesian Change Point Detection algorithm. The algorithm may use Bayesian statistics to detect change point in data, by comparing the likelihood of the data under different hypothesis about the change point. Examples of machine learning being used to fuse sensor data readings together are provided in U.S. Pat. No. 11,506,606, issued on Nov. 22, 2022, and entitled “Alarm Threshold Organic And Microbial Fluorimeter And Methods,” and U.S. Pat. No. 11,507,861, issued on Nov. 22, 2022, and entitled “Machine Learning Techniques For Improved Water Conversation And Management.” Both of these patents are incorporated herein in their entireties.

The system 300 may utilize carbon sensors, temperature sensors, moisture sensors, decomposition sensors, and accumulation sensors, of which outputs are processed to identify vertical-direction step changes or vertical gradients in soil process monitoring to provide additional mechanistic constraints on model output (via data assimilation algorithms) to generate repeatedly-updated estimates of in field carbon sequestration.

The system 300 may rely on a data assimilation and modeling package to estimate soil carbon sequestration in an agricultural field. The data generated by each of the sensors of the carbon sensor devices 370-1-370-n in the system 300 alone may not individually yield good estimates of soil carbon changes. To achieve this measurement, the data stream from the sensors of the carbon sensor devices 370-1-370-n may be fed to a series of soil decomposition and stabilization algorithms using a data assimilation framework that allows for continuous model correction and adjustment.

The analytic output from the carbon modeling and data assimilation package implemented in the system 300 may include past rates of carbon accumulation and uncertainty estimations, current assimilation rates, and forecasts of future carbon sequestration based on field trends and data flows. Further, due to long lasting characteristics and low costs of the carbon sensing devices 370-1-370-n, a large number of the carbon sensing devices 370-1-370-n may be planted on the region of interest, it is possible to document changes in soil carbon stocks on an annual basis-which is the timescale for issuing carbon credits-because the annual rate of change in soil carbon was indetectable with conventional methods.

The system 300 may include a computing device 310, which has an input device 320, a display 330, a processor 340, a memory 350, and a network interface 360. The computing device 310 may be a stand-alone server, a network server, a cloud server, or a software as a service (“SaaS”). The computing device 310 may collect or aggregate the sensor outputs from the carbon sensor devices 370-1-370-n via the network interface 360.

Collection or aggregation of sensor outputs may be performed via a wired or wireless connection. In an aspect, output collectors may transmit radiofrequency signals to activate or power the carbon sensor devices 370-1-370-n and receive the sensor outputs therefrom. In an aspect, a near field communication (NFC) protocol may be followed to maintain a high level of security. For example, output collectors may bring an NFC transceiver and approach to each of the carbon sensor devices 370-1-370-n within 20 cm or 5 cm to receive sensor outputs therefrom.

In another aspect, a radio frequency identification (RFID) may be used for collecting the sensor outputs. For example, an active RFID may reach 1,500 feet for connection and a passive RFID may reach 20 feet. Thus, compared to the NFC protocol, the network interface 360 using the RFID protocol may be used to reach one or more carbon sensor devices 370-1-370-n. Thus, based on the range reachable by the network interface 360 and the number of the carbon sensor devices in the range, the output collector may determine how many places to visit the plot of land. After collecting the sensor outputs from the carbon sensor devices 370-1-370-n, the sensor outputs may be transmitted to the system 300 via the network interface 360.

The system 300 may preprocess the sensor outputs by performing one or more programs or algorithms, which are computer-executable instructions saved on the memory 350 and executed by the processor 340. The system 300 may utilize one or more models related to estimating carbon sequestration, or in general soil health.

In an aspect, one or more models may include a soil carbon pool model with pool-to-pool transfers, representation of mineral stabilization fluxes, pool specific decomposition rates, and/or moisture and temperature controls. In another aspect, the models may include implementation of a matrix solution for the coupled equations with diagonal matrices representing the fundamental controls on carbon decomposition, temperature, and moisture controls, stabilization transfer coefficients, etc. This formulation may be well-suited to the subsequent data assimilation. In still another aspect, the models may include usage of optimal data assimilation approaches using exploratory data (soil CO2, moisture, and temperature) from the NEON observatory sites for testing of approaches and development of initial parameter sets. Completion of coupled matrix and data assimilation package for testing with laboratory data. In a further aspect, the models may include a comparison of forward modeled projections of carbon storage and loss against observed laboratory data. In a still further aspect, the models may include testing of full data assimilation framework with laboratory data generated from sensor experiments.

While the gradient flux method has been deployed successfully in the field to measure CO₂ efflux, the sensors used for the required CO₂ and soil moisture measurements have traditionally been expensive and decoupled, requiring large investments for both sensors and datalogging platforms. On the other hand, the low-cost NDIR and capacitive moisture sensors used in the carbon sensor devices 370-1-370-n may make them suitable for in-situ soil deployments. In an aspect, an individual sensor device 370-1-370-n may be equipped with a carbon sensor, a temperature sensor, a moisture sensor, a carbon accumulation sensor, and a decomposition sensor. Further, the carbon sensor device 370-1-370-n includes a plurality of arrays of carbon-related sensors, and each array may be affixed to the carbon sensor device for modular depth measurements.

In an aspect, the system 300 may perform an effective weatherization process to ruggedize low-cost CO₂ sensors. Weatherization process may build on previous literature and incorporate hybrid additive manufacturing approaches to make the process rapid and repeatable. Additionally, in another aspect, the system 300 may perform validation of sensor response time and accuracy as benchmarked by reference and sensors. Validation may be performed ruggedly under high moisture conditions. In addition, the system 300 may be geometrically optimized timer- or touch sensor-based capacitive soil moisture sensing platform. In another aspect, the system 300 may perform validation of soil moisture sensor in laboratory conditions. Performance may be compared to existing low-cost capacitive platforms already examined in the literature, e.g., the SEN0193 sensor. Soil-or site-specific calibration requirements may be examined in controlled settings/Soil settling time and strategies may be examined.

A number of protocols (CAR SEP, VM0042, FAO GSOC, Gold Standard's SOC Framework Methodology and BCarbon) may combine process-based models with direct field measurements of SOC to verify model predictions. In an aspect, the system 300 may provide an additional measurement and data assimilation/modeling approach to these methodologies. The system 300 may present an opportunity to verify models, secure long-term carbon credit verifications, and provide information on spatial variability of SOC over large sites using high spatial resolution in a Phase 2 proposal, providing opportunities for improved market support by reducing risks in SOC credit purchases.

Carbon registries and private companies have developed SOC verification protocols to bring carbon credits to the market and pay farmers for sequestering carbon. Farmers may be able to sell these credits to companies for use in voluntary carbon markets as part of corporate sustainability efforts or in compliance markets to meet climate mitigation targets. Current carbon verifications that include carbon sequestration are Verified Carbon Standard (VCS), American Carbon Registry, Climate Action Reserve (CAR), Plan Vivo, Alberta Carbon Offset System, and Australia Emissions Reduction Fund (ERF). From a voluntary perspective, the number of commitments from corporations and governments to net-zero and carbon positive climate targets are on the rise. The Paris Climate Agreement's focus on voluntary markets has led to a surge in independent systems with voluntary credits reaching 65% of total annual credits issued in 2019, compared to only 17 percent in 2015. Thus, based on the carbon sequestration estimated by the system 300, the owners of lands or fields may utilize the carbon credit with credibility.

The system 300 may utilize artificial intelligence (AI) or machine learning (ML) to update/modify/enhance one or more models to estimate carbon sequestration. Addition to the sensor outputs, the system 300 may use environmental parameters in estimating carbon sequestration. Specifically, the sensor outputs generated by the carbon sensor devices 370-1-370-n may not provide quantitative values of CO₂, temperature, and moisture content. Instead, the sensor outputs generated by the carbon sensor devices 370-1-370-n may provide trends of changes in CO₂, temperature, and moisture content along the vertical direction from the soil to the air. In other words, the system 300 may not be able to provide quantitative values of carbon sequestration only with the sensor outputs. However, the system 300 may be able to provide quantitative values of carbon sequestration based on the sensor outputs and the environmental parameters.

The environmental parameters may include geospatial data, which includes the location and elevation of the region of interest, and weather conditions, which include temperature, pressure, moisture, in the environment or air at the site where the carbon sensor devices 370-1-370-n are planted. In an aspect, the environmental parameters may also include net primary product (total carbon produced in biomass) by farmers. In another aspect, the system 300 may also use history information about the carbon sequestration. By using the sensor outputs, the environmental parameters, and/or history information, the system 300 may train AI or ML algorithms to estimate the carbon sequestration. Thereby, the system 300 may generate one or more models. Further, the one or more trained models may be able to estimate carbon sequestration based on only with the sensor outputs and the environmental parameters.

In an aspect, the system 300 may further update/refine/modify the one or more models to provide more accurate estimations of the carbon sequestration. In this regard, the AI and ML algorithms may be further trained based unsupervised or supervised manner with or without reinforcement with constant addition of sensor outputs and the environmental parameters.

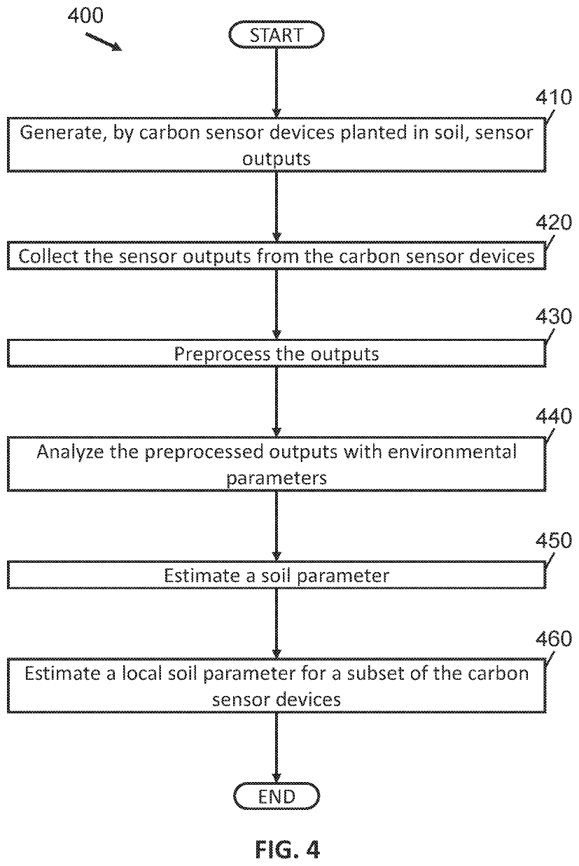

Now returning to FIG. 4, illustrated is a flowchart of a method 400 for predicting and estimating parameters relating to soil health according to aspects of the present disclosure. As illustrated in FIG. 3, the plurality of carbon sensor devices may be planted or inserted into the soil of the region of interest. The placements of the carbon sensor devices may be determined based on representative characteristics of the region of interest for the prediction and estimation of the soil health.

At step 410, the carbon sensor devices may generate sensor outputs. The sensor outputs may include sensed results related to carbon, such as temperature, moisture, carbon, carbon accumulation, and/or microbial decomposition. In an aspect, the carbon sensor devices may have vertical placements of arrays of sensors. In other words, each array may include a temperature sensor, a carbon sensor, a moisture sensor, a carbon accumulation sensor, and a microbial decomposition sensor, and arrays are disposed at different places along the vertical direction. Further, the arrays of sensors are all inserted into the soil. Thus, the sensor outputs may provide vertical gradients of temperature, moisture, carbon, carbon accumulation, and/or microbial decomposition in the soil.

Due to the low cost sensors in each array, the sensor outputs may not provide definite quantitative values of temperature, moisture, carbon, carbon accumulation, and/or microbial decomposition. Rather, relative changes or trend of changes may be identified by the sensor outputs. The sensor outputs directly from the sensors are generally analog. In an aspect, the carbon sensor devices may include an analog-to-digital converter (ADC), which converts the analog sensor outputs to digital sensor outputs, and store the digital sensor outputs in a memory.

At step 420, the sensor outputs or the digital sensor outputs may be collected by following a wired or wireless communication protocol by a computing device (e.g., the computing device 310 of FIG. 3). The collection frequency may be once daily, weekly, bi-weekly, monthly, bi-monthly, quarterly, semi-annually, or annually based on predetermined requirements related to characteristics of the region of interest.

The sensor outputs may include identification information of each carbon sensor device and each array of sensor thereof. For example, in a configuration that each carbon sensor device has three arrays of sensors along the vertical or longitudinal direction thereof, identification of their positions (e.g., top, middle, and bottom) may be included in the identification information together with the respective sensor outputs. Also, the identification information may include information about which carbon sensor device the sensor outputs are from.

In an aspect, environmental parameters may be also collected at step 420. For example, the environmental parameters may include temperature, pressure, or moisture in the air or environment. In another aspect, the environmental parameters may include geospatial data related to the location and elevation of the region of interest. To calculate carbon sequestration, the owner or farmer of the region of interest may need to provide the total carbon produced in biomass. Thus, when the farmer grows different crops four times a year, the total carbon produced in biomass in the crops may be collected four times a year at step 420.

At step 430, the computing device may preprocess the sensor outputs by eliminating outliers in the sensor outputs. Unexpected or sudden hikes or anomalies in the trend of changes in temperature, moisture, carbon, carbon accumulation, and/or microbial decomposition may be removed at step 430. In a case where the unexpected or sudden hikes or anomalies in the trend are continuously maintained, that signals that the carbon sensor device may run out its lifespan or needs to be replaced or repaired. The computing device may be able to provide to the operator or the farmer a notification of error at the identified carbon sensor device based on the anomalies.

The computing device may process or analyze the preprocessed outputs with the environmental parameters at step 440. In particular, a machine learning (ML) or artificial intelligence (AI) algorithm may be employed in the analysis. The ML or AI algorithm may have been trained with history data of previous sensor outputs, environmental parameters, and carbon sequestration. The AI and ML algorithm may be further trained based unsupervised or supervised manner with or without reinforcement with constant addition of sensor outputs and the environmental parameters.

Specifically, sensor outputs from one carbon sensor device may include three sets of sensor outputs when the carbon sensor device includes three arrays of sensors. Based on these three sets of sensor outputs, the computing device may be able to calculate vertical gradients of temperature, moisture, carbon, carbon accumulation, and/or microbial decomposition.

Based on aggregate vertical gradients from all of the carbon sensor devices, the ML and AI algorithm of the computing device may be able to predict or estimate a soil parameter of the region of interest with the environmental parameters at step 450. The soil parameter may be carbon sequestration. Further, based on the carbon sequestration, the computing device may be able to estimate net flux of CO₂ between soil CO₂ fluxes and the net primary production (total carbon produced in biomass) of the plants so as to determine whether the region of interest is accumulating or losing carbon. In a case where the region of interest is accumulating carbon, the owner or farmer of the region of interest may receive financial benefits by selling the accumulated carbon to individuals or businesses who need carbon credits to compensate for their unavoidable emissions of carbon.

Steps 410-450 may be considered as upscaling because sensor outputs from individual carbon sensor devices are used to produce estimation of the aggregate carbon sequestration. After the aggregate carbon sequestration of the region of interest is estimated, a local soil parameter for each carbon sensor device or a subset of the carbon sensor devices may be estimated based on the aggregate carbon sequestration. The local soil parameter may be local carbon sequestration. Since the aggregate carbon sequestration is used to estimate local carbon sequestration, step 460 may be considered as downscaling. Based on the aggregate or local carbon sequestration, general or local soil health may be predicted, respectively.

Further, based on estimated local carbon sequestration, a carbon credit map may be generated for the region of interest. In a case where anomalies are identified in the carbon credit map, appropriate measure (e.g., replacement of the carbon sensor device or addition of fertilizer) may be taken to address the anomalies.

### Example Implementations

In view of the foregoing, the present invention relates, for example and without being limited thereto, to the following aspects:

In a first aspect, a carbon sensor device is configured for sensing changes in carbon sequestration in soil. The carbon sensor device includes an elongated body configured to be planted into soil along a longitudinal direction thereof, and a plurality of arrays of sensors fixedly attached to the elongated body, wherein an array of sensors includes a carbon dioxide sensor, a moisture sensor, and a temperature sensor. The plurality of arrays of sensors are attached to different positions along the longitudinal direction.

In a second aspect of the carbon sensor device as recited in any of the preceding aspects, the plurality of arrays of sensors further includes a carbon accumulation sensor and a carbon decomposition sensor.

In a third aspect of the carbon sensor device as recited in any of the preceding aspects, the carbon sensor device is to be planted into the soil by 1 meter from a surface of the soil.

In a fourth aspect of the carbon sensor device as recited in any of the preceding aspects, the carbon sensor device is to be planted into the soil by 50 centimeters from a surface of the soil.

In a fifth aspect of the carbon sensor device as recited in any of the preceding aspects, outputs generated by the plurality of arrays of sensors provide vertical gradients of temperature, moisture, and carbon dioxide toward a surface of the soil.

In a sixth aspect of the carbon sensor device as recited in any of the preceding aspects, the carbon sensor device further includes a memory configured to store outputs generated by the plurality of arrays of sensors, and a network interface configured to transmit the outputs generated by the plurality of arrays of sensors.

In a seventh aspect of the carbon sensor device as recited in any of the preceding aspects, the network interface transmits the outputs following a near field communication, Bluetooth, or wireless protocol.

In an eighth aspect of the carbon sensor device as recited in any of the preceding aspects, the carbon sensor device further includes a memory configured to store identification information including a location, where the carbon sensor device is planted, or a unique identification of the carbon sensor device.

In a ninth aspect of the carbon sensor device as recited in any of the preceding aspects, the carbon sensor device further includes a power source configured to supply power to the plurality of arrays of sensors.

In a tenth aspect of the carbon sensor device as recited in any of the preceding aspects, the power source is a battery, a renewable power source, or an antenna, which receives power via radiofrequency signals.

In an eleventh aspect, a system is configured for detection and monitoring of soil carbon sequestration. The system includes a plurality of carbon sensor devices according to the first aspect, each being configured to be planted in soil at a plurality of sites, a network interface configured to aggregate outputs generated by the plurality of carbon sensor devices, one or more processors, and one or more computer-readable media having stored thereon executable instructions that, when executed by the one or more processors, configure the system to perform: preprocessing the aggregated outputs, analyzing, by a machine learning algorithm, the preprocessed outputs with environmental parameters, estimating a soil parameter of the plurality of sites, and estimating a local soil parameter for at least a subset of the plurality of sensor sites based on the estimated soil parameter.

In a twelfth aspect of the system as recited in any of the preceding aspect from the eleventh aspect, the plurality of arrays of sensors further includes a carbon accumulation sensor and a carbon decomposition sensor.

In a thirteenth aspect of the system as recited in any of the preceding aspect from the eleventh aspect, the plurality of carbon sensor devices are to be planted into the soil by 1 meter from a surface of the soil.

In a fourteenth aspect of the system as recited in any of the preceding aspect from the eleventh aspect, the carbon sensor device is to be planted into the soil by 50 centimeters from a surface of the soil.

In a fifteenth aspect of the system as recited in any of the preceding aspect from the eleventh aspect, the outputs generated by the plurality of arrays of sensors provide vertical gradients of temperature, moisture, and carbon dioxide toward a surface of the soil.

In a sixteenth aspect of the system as recited in any of the preceding aspect from the eleventh aspect, each carbon sensor device transmits respective outputs to the network interface following a near field communication, Bluetooth, or wireless protocol.

In a seventeenth aspect of the system as recited in any of the preceding aspect from the eleventh aspect, the environmental parameters include a weather, temperature, moistures, pressure, or geospatial data of the plurality of sites.

In an eighteenth aspect of the system as recited in any of the preceding aspect from the eleventh aspect, the soil parameter is related to soil health.

In a nineteenth aspect of the system as recited in any of the preceding aspect from the eleventh aspect, the soil parameter includes an amount of carbon sequestration from the soil to air.

In a twentieth aspect of the system as recited in any of the preceding aspect from the eleventh aspect, a computer-implemented method is configured for estimating soil health. The method includes aggregating outputs generated by a plurality of carbon sensor devices, of which each according to clause 1 is configured to be planted in soil at a respective one of a plurality of sites, preprocessing the aggregated outputs, analyzing, by a machine learning algorithm, the preprocessed outputs with environmental parameters, estimating a soil parameter of the plurality of sites, and estimating a local soil parameter for at least a subset of the plurality of sensor sites based on the estimated soil parameter.

Further, the methods may be practiced by a computer system including one or more processors and computer-readable media such as computer memory. In particular, the computer memory may store computer-executable instructions that when executed by one or more processors cause various functions to be performed, such as the acts recited in the embodiments.

Computing system functionality can be enhanced by a computing systems' ability to be interconnected to other computing systems via network connections. Network connections may include, but are not limited to, connections via wired or wireless Ethernet, cellular connections, or even computer to computer connections through serial, parallel, USB, or other connections. The connections allow a computing system to access services at other computing systems and to receive application data quickly and efficiently from other computing systems.

Interconnection of computing systems has facilitated distributed computing systems, such as so-called “cloud” computing systems. In this description, “cloud computing” may be systems or resources for enabling ubiquitous, convenient, on-demand network access to a shared pool of configurable computing resources (e.g., networks, servers, storage, applications, services, etc.) that can be provisioned and released with reduced management effort or service provider interaction. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Cloud and remote based service applications are prevalent. Such applications are hosted on public and private remote systems such as clouds and usually offer a set of web-based services for communicating back and forth with clients.

Many computers are intended to be used by direct user interaction with the computer. As such, computers have input hardware and software user interfaces to facilitate user interaction. For example, a modern general-purpose computer may include a keyboard, mouse, touchpad, camera, etc. for allowing a user to input data into the computer. In addition, various software user interfaces may be available.

Examples of software user interfaces include graphical user interfaces, text command line-based user interface, function key or hot key user interfaces, and the like.

Disclosed embodiments may comprise or utilize a special purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general purpose or special purpose computer system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: physical computer-readable storage media and transmission computer-readable media.

Physical computer-readable storage media includes RAM, ROM, EEPROM, CD-ROM or other optical disk storage (such as CDs, DVDs, etc.), magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry program code in the form of computer-executable instructions or data structures, and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, and the like. The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A carbon sensor device for sensing changes in carbon sequestration in soil, the carbon sensor device comprising:
an elongated body configured to be planted into soil along a longitudinal direction thereof; and
a plurality of arrays of sensors fixedly attached to the elongated body,
wherein an array of sensors includes a carbon dioxide sensor, a moisture sensor, and a temperature sensor, and
wherein the plurality of arrays of sensors are attached to different positions along the longitudinal direction.

2. The carbon sensor device according to claim 1, wherein the plurality of arrays of sensors further includes a carbon accumulation sensor and a carbon decomposition sensor.

3. The carbon sensor device according to claim 1, wherein the carbon sensor device is to be planted into the soil by 1 meter from a surface of the soil.

4. The carbon sensor device according to claim 1, wherein the carbon sensor device is to be planted into the soil by 50 centimeters from a surface of the soil.

5. The carbon sensor device according to claim 1, wherein outputs generated by the plurality of arrays of sensors provide vertical gradients of temperature, moisture, and carbon dioxide toward a surface of the soil.

6. The carbon sensor device according to claim 1, further comprising:
a memory configured to store outputs generated by the plurality of arrays of sensors; and
a network interface configured to transmit the outputs generated by the plurality of arrays of sensors.

7. The carbon sensor device according to claim 6, wherein the network interface transmits the outputs following a near field communication, Bluetooth, or wireless protocol.

8. The carbon sensor device according to claim 1, further comprising:
a memory configured to store identification information including a location, where the carbon sensor device is planted, or a unique identification of the carbon sensor device.

9. The carbon sensor device according to claim 1, further comprising:
a power source configured to supply power to the plurality of arrays of sensors.

10. The carbon sensor device according to claim 9, wherein the power source is a battery, a renewable power source, or an antenna, which receives power via radiofrequency signals.

11. A system for detection and monitoring of soil carbon sequestration, the system comprising:
a plurality of carbon sensor devices according to claim 1, each being configured to be planted in soil at a plurality of sites;
a network interface configured to aggregate outputs generated by the plurality of carbon sensor devices;
one or more processors; and
one or more computer-readable media having stored thereon executable instructions that, when executed by the one or more processors, configure the system to perform:
preprocessing the aggregated outputs;
analyzing, by a machine learning algorithm, the preprocessed outputs with environmental parameters;
estimating a soil parameter of the plurality of sites; and
estimating a local soil parameter for at least a subset of the plurality of sensor sites based on the estimated soil parameter.

12. The system according to claim 11, wherein the plurality of arrays of sensors further includes a carbon accumulation sensor and a carbon decomposition sensor.

13. The system according to claim 11, wherein the plurality of carbon sensor devices are to be planted into the soil by 1 meter from a surface of the soil.

14. The system according to claim 11, wherein the carbon sensor device is to be planted into the soil by 50 centimeters from a surface of the soil.

15. The system according to claim 11, wherein the outputs generated by the plurality of arrays of sensors provide vertical gradients of temperature, moisture, and carbon dioxide toward a surface of the soil.

16. The system according to claim 11, wherein each carbon sensor device transmits respective outputs to the network interface following a near field communication, Bluetooth, or wireless protocol.

17. The system according to claim 11, wherein the environmental parameters include a weather, temperature, moistures, pressure, or geospatial data of the plurality of sites.

18. The system according to claim 11, wherein the soil parameter is related to soil health.

19. The system according to claim 11, wherein the soil parameter includes an amount of carbon sequestration from the soil to air.

20. A computer-implemented method for estimating soil health comprising:
aggregating outputs generated by a plurality of carbon sensor devices, of which each according to claim 1 is configured to be planted in soil at a respective one of a plurality of sites;
preprocessing the aggregated outputs;
analyzing, by a machine learning algorithm, the preprocessed outputs with environmental parameters;
estimating a soil parameter of the plurality of sites; and
estimating a local soil parameter for at least a subset of the plurality of sensor sites based on the estimated soil parameter.
